SEQUENCE LISTING (8 sequences)

SEQ ID NO: 1 - protein, 4 aa, synthetic construct
RRRG

SEQ ID NO: 2 - protein, 5 aa, synthetic construct
KFERQ

SEQ ID NO: 3 - protein, 11 aa, synthetic construct
YGRKKRRQRRR

SEQ ID NO: 4 - DNA, 33 bp, synthetic construct
  source 1..33: mol_type = other DNA
tatggcaggaagaagcggagacagcgacgaaga

SEQ ID NO: 5 - protein, 25 aa, synthetic construct
YGRKKRRQRRRRTKSGVYLVGRRRG

SEQ ID NO: 6 - DNA, 42 bp, synthetic construct
  source 1..42: mol_type = other DNA
cgtactaaatctggtgtttatttggttggtcgacgacgaggc

SEQ ID NO: 7 - protein, 26 aa, synthetic construct
YGRKKRRQRRRRTKSGVYLVGKFERQ

SEQ ID NO: 8 - DNA, 81 bp, synthetic construct
  source 1..81: mol_type = other DNA
atgtatggcaggaagaagcggagacagcgacgaagacgtactaaatctggtgtttatttggttggtcgacgacgaggctga

(19) United States
(12) Patent Application Publication
(10) Pub. No.: US 2026/0248967 A1
(43) Pub. Date: Aug. 27, 2026

(54) AAV VECTOR AND USE THEREOF

(71) Applicant: PRODEGRE THERAPEUTICS (SHANGHA) CO., LTD., Shanghai (CN)

(72) Inventors: Xin Yang, Shenzhen (CN); Tian Tian, Shenzhen (CN); Yu Tian Wang, Shenzhen (CN)

(21) Appl. No.: 18/877,203

(22) PCT Filed: May 11, 2023
(86) PCT No.: PCT/CN2023/093379

§ 371 Date: Dec. 19, 2024

(51) Int. Cl. A61K 48/00; A61K 9/00; A61P 25/28; C07K 14/47; C12N 15/86

(52) U.S. Cl. CPC A61K 48/0058; A61K 9/0085; A61K 48/0075; A61P 25/28; C07K 14/4711; C12N 15/86; C07K 2319/10; C07K 2319/95; C12N 2750/14143

(57) ABSTRACT

The present invention provides an adeno-associated virus (AAV) vector and use thereof. The AAV vector comprises a nucleic acid fragment encoding a secreted tat-degradation peptide. The AAV vector designed in the present invention can be continuously expressed in the peripheral nervous system, where the expressed degradation peptides can cross the blood-brain barrier and cell membranes to exert long-term therapeutic effects in central nervous system (CNS) diseases. Additionally, the vector can also be continuously expressed in the CNS, where the degradation peptides cross the cell membrane to mediate its effects in both AAV-infected and non-infected cells. Therefore, the present invention offers an AAV vector that enables widespread and long-acting delivery of a therapeutic degradation peptide.

### TECHNICAL FIELD

The present invention presents an adeno-associated virus (AAV) vector and use thereof, pertaining to the field of AAV drugs.

### BACKGROUND

Traditional polypeptide drugs, particularly those made from natural amino acids, are rapidly degraded in the body, resulting in a short duration of effective drug concentration that hinders the attainment of desired therapeutic effects. Furthermore, traditional polypeptide drugs cannot be orally administrated and require repeated intramuscular injections. In addition, traditional polypeptide drugs often fail to efficiently cross the blood-brain barrier (BBB), limiting their ability to mediate therapeutic effects in the central nervous system (CNS). Traditional degradation peptides are predominantly used for acute treatment (such as stroke) in clinical practice. However, these therapeutics are difficult to maintain at effective drug concentrations in the body, exhibit suboptimal efficacy in chronic diseases, and lead to poor patient compliance due to the need for long-term injections.

Given the challenges associated with natural polypeptide drugs, such as rapid degradation in the body leading to short maintenance of effective drug concentrations, difficulties in achieving consistent efficacy, and relatively high individual variability, adeno-associated virus (AAV) therapy may serve as a promising alternative for clinical use. This technology centers on using an AAV vector to deliver a target gene, which is then expressed in the infected cells to restore the protein that is deficient or missing. A single dose of an AAV vector drug is expected to remain effective for 5 to 10 years. However, limitations of this technology include: 1) low infection efficiency and a therapeutic effect that is confined to only a small number of infected cells; 2) Traditional AAV vectors and their encoded proteins are unable to cross the BBB and cell membrane, preventing widespread therapeutic effects across infected cells, the brain, and the peripheral nervous system.

### SUMMARY

To address the problems in the prior art, an AAV vector and use thereof are proposed.

The present invention provides an AAV vector comprising a nucleic acid fragment that encodes a tat-degradation peptide.

The degradation peptide of the present invention refers to a peptide that mediates targeted degradation of endogenous proteins.

The tat-degradation peptide of the present invention is a secreted tat-degradation peptide. The AAV vector designed in the present invention can be continuously expressed in the periphery, where the expressed degradation peptides can cross the BBB and cell membranes to exert long-term therapeutic effects in CNS diseases. Additionally, the vector can also be continuously expressed in the CNS, where the degradation peptides cross the cell membrane to mediate its effects in both AAV-infected and non-infected cells. Therefore, the present invention offers an AAV vector that enables widespread and long-acting delivery of a therapeutic degradation peptide.

The tat of the present invention refers to the human immunodeficiency virus-1 transcription activator (HIV-1 tat), which is a nontoxic and highly cell-permeable peptide. In a particular embodiment, preferably, the degradation peptide is a peptide carrying a deg tag or a CTM tag that mediates endogenous protein degradation;
preferably, the amino acid sequence of the deg tag is as shown in SEQ ID NO. 1;

SEQ ID NO.1:
RRRG;

preferably, the amino acid sequence of the CTM tag is as shown in SEQ ID NO. 2;

SEQ ID NO.2:
KFERQ.

In a particular embodiment, preferably, the amino acid sequence of the tat is as shown in SEQ ID NO. 3;

SEQ ID NO.3:
YGRKKRRQRRR;

preferably, the nucleic acid sequence encoding tat is as shown in SEQ ID NO. 4;

SEQ ID NO.2:
TATGGCAGGAAGAAGCGGAGACAGCGACGAAGA.

In a particular embodiment, preferably, the degradation peptide is βSyn-deg; and
the amino acid sequence of βSyn-deg is as shown in SEQ ID NO. 5;

SEQ ID NO.5:
YGRKKRRQRRRRTKSGVYLVGRRRG;

preferably, the nucleic acid sequence encoding βSyn-deg is as shown in SEQ ID NO. 6;

SEQ ID NO.6:
CGTACTAAATCTGGTGTTTATTTGGTTGGTCGACGACGAGGC.

The deg of the present invention, also known as degron, is a component of the tat-βsyn-degron construct that mediates the binding of α-synuclein (α-syn) to the proteasome, thereby inducing its degradation. The tat-βsyn-degron of the present invention can effectively and specifically knockdown α-syn.

Under physiological conditions, there are three types of synuclein: α-syn, β-synuclein (β-syn), and γ-synuclein (γ-syn). α-syn exists as a disordered monomer under physiological conditions but can aggregate into a toxic form under pathological conditions. The secreted tat-degradation peptide of the present invention recognizes and binds to α-syn through the encoded β-syn sequence, mediating α-syn degradation.

In a particular embodiment, preferably, the degradation peptide is βsyn-CTM; and the amino acid sequence of βsyn-CTM is as shown in SEQ ID NO. 7;

SEQ ID NO.7:
YGRKKRRQRRRRTKSGVYLVGKFERQ.

In a particular embodiment, preferably, the AAV vector comprises a promoter sequence; preferably, the promoter is a CMV promoter or a cell-specific promoter;
preferably, the cell-specific promoter is EF1a or hSyn.

In a particular embodiment, the serotype of the AAV vector is AAV9, AAV2, AAV5 or AAV8. The present invention does not explicitly limit the AAV serotype used, and the corresponding serotypes are used based on the sites of injection in practical application. For example, AAV9 can be selected for peripheral injection due to its capacity to cross the BBB, whereas AAV2, AAV5, or AAV8 is preferred for local CNS injection due to their inability to cross this barrier.

The second aspect of the present invention provides use of the AAV vector in preparing a drug for intervention in CNS diseases. The drug exhibits widespread and sustained interventional efficacy in CNS diseases.

The third aspect of the present invention provides a drug comprising the AAV vector and pharmaceutically acceptable excipients.

In a particular embodiment, preferably, the drug is administered as an injection; and more preferably, the drug is an intracerebral injection.

The beneficial effects of the present invention are:
1. The AAV vector of the present invention comprises a nucleic acid fragment that encodes a tat-degradation peptide, demonstrating higher infection efficiency and a broader targeting effect on infected cells compared to traditional nucleic acid interventions. Cells infected with the AAV vector continuously secrete the degradation peptide, which can cross both the BBB and cell membrane, allowing for widespread delivery and long-term intervention. In practical application, the AAV vector of the present invention effectively overcomes the limitations of traditional degradation peptide drugs, including the difficulty in maintaining effective drug concentrations in the body, suboptimal therapeutic efficacy in chronic diseases, and poor patient compliance due to the need of long-term injection. The present invention offers an AAV vector that enables widespread and long-acting delivery of a traditional degradation peptide, thereby enhancing its practical application.
2. The AAV vector of the present invention can be injected at specific sites during brain surgery to enable the continuous expression of the degradation peptide, which then crosses the cell membrane and the BBB to mediate its therapeutic effects. Therefore, the AAV vector of the present invention allows for injection in a single brain region while generating effects across multiple brain regions.
3. The AAV vector of the present invention can express the target gene in the host for extended periods, allowing for long-term CNS intervention from a single administration. This addresses the limitations of both traditional AAV vectors, which only act on transfected cells and are ineffective in treating CNS diseases, and natural polypeptide drugs, which degrade rapidly in the body, making it difficult to maintain effective drug concentrations and leading to poor efficacy.
4. The AAV vector of the present invention represents an improvement over traditional AAV vectors and degradation peptide therapies. It retains the advantages of conventional AAV vector therapy, including a favorable safety profile, therapeutic potential, long-term efficacy, and clinical feasibility, while also incorporating the genetically modifiable characteristics of polypeptide drugs.

### BRIEF DESCRIPTIONS

FIG. 1 is a diagram of the vector after insertion of the nucleic acid fragment containing tat-βsyn-degron in this example.

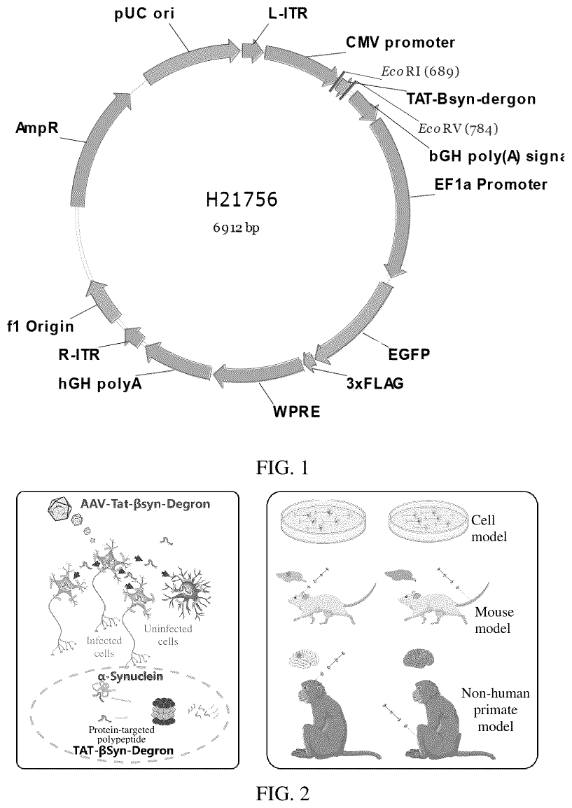

FIG. 2 demonstrates the pattern of expression of the AAV vector constructed in example 1 in animal models.

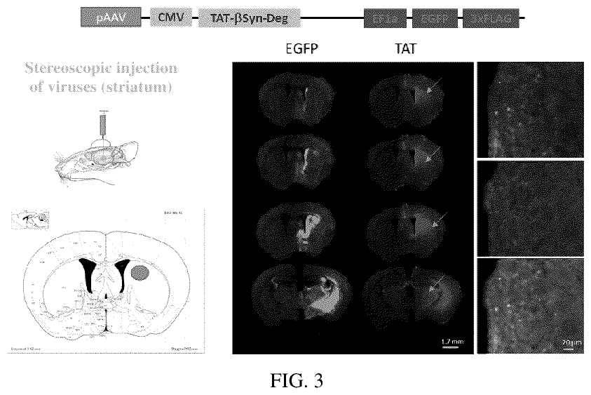

FIG. 3 shows the sites and coordinates for intracerebral injection in experimental example 1, and the right panel in FIG. 3 shows the expression and distribution of green fluorescent proteins and tat.

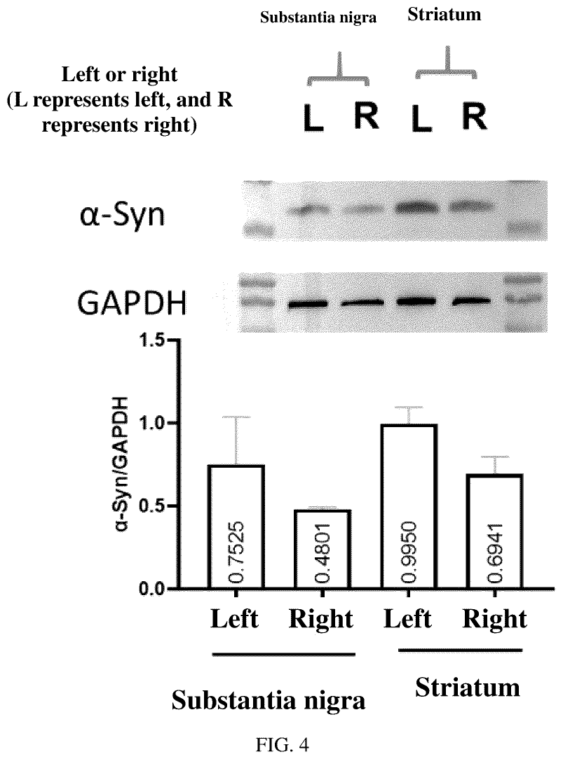

FIG. 4 shows the levels of α-syn in the right substantia nigra and striatum in a mouse model of Parkinson's disease at 3 months post-injection.

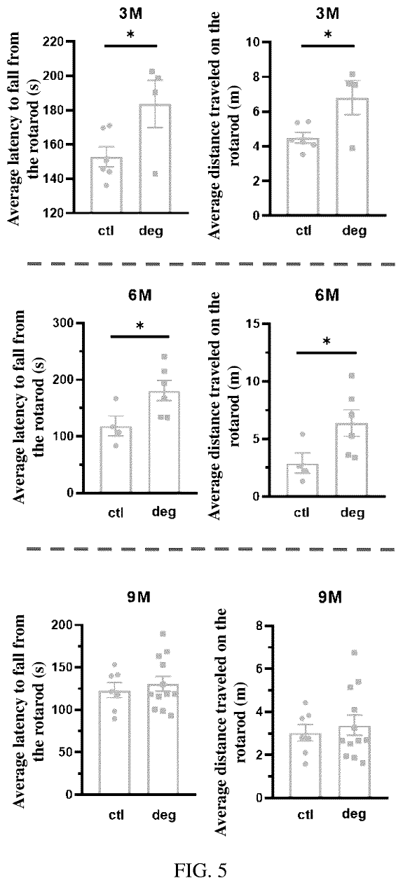

FIG. 5 presents the latency to fall and distance traveled by mice with Parkinson's disease on a rotarod at 3 months post-injection.

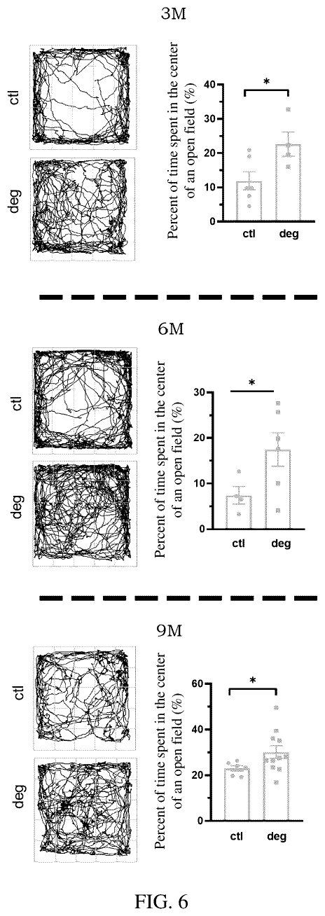

FIG. 6 shows the time spent in the center of an open field by mice with Parkinson's disease at 3 months post-injection.

### DETAILED EMBODIMENTS

To clarify the technical features, purposes, and benefits of the present invention, the technical solution will be described in detail; however, this description should not be construed as limiting the embodiments of the invention.

### Example 1

This example provides a method for constructing an AAV vector comprising a tat-βsyn-degron construct. The method comprises:

(1) In vitro synthesis of the nucleic acid fragment encoding tat-βsyn-degron. The sequence is as follows: SEQ ID NO. 8: ATGTATGGCAGGAAGAAGCGGAGACAGCGACGAAGACGTACTAAATCTGGTGT TTATTTGGTTGGTCGACGACGAGGCTGA.

### (2) Construction of Expression Vector

Serotype AAV2/9, which causes localized brain infection, is used to construct an AAV expression vector (also known as pAAV), where the CMV promoter drives the transcription and expression of tat-βSyn-degron.

FIG. 1 is a diagram of the vector after insertion of the nucleic acid fragment containing tat-βsyn-degron in this example. The structure of the vector is pAAV-CMV-tat-βSyn-Deg-ef1a-egfp-3×flag. The virus can be purchased from OBiO Technology.

### Experimental Example 1

This experimental example provides the transmembrane expression of the AAV vector constructed in example 1 in animal models.

The pattern of expression of the constructed AAV vector in example 1 in animal models is shown in FIG. 2. In FIG. 2, the left panel shows that transfected and infected cells in the brain can continuously express and secrete pharmacologically active tat-βsyn-deg degradation peptides after local intracerebral injection or intravenous injection of the AAV vector (the method of injection is shown in the right panel of FIG. 2). The degradation peptide is secreted by the host cells via the tat transmembrane sequence and enters infected or uninfected cells in the periphery to exert its therapeutic effects. Since the AAV vector can support prolonged target gene expression, this system provides extended efficacy and functions as an in vivo intracerebral pharmaceutical factory.

As shown in FIG. 3, on day 30 post-injection of 1 μL of 2×10¹² v.g./mL pAAV in mouse striatum, perfusion fixation of the brain followed by tat staining showed high EGFP expression within the striatum and widespread tat distribution beyond EGFP-positive host cells due to its transmembrane diffusion capability. The enlarged images in the right panel of FIG. 3 demonstrate that tat (red fluorescence) was generated by green fluorescent cells and diffused outward. These data demonstrate the feasibility of the technical solution of the in vivo pharmaceutical factory.

### Experimental Example 2

This experimental example provides the effective expression of the AAV vector constructed in example 1 in animal model.

Research has shown that tat-βSyn-deg knocks down α-syn protein expression in a targeted manner. However, because the effects of current tat-βSyn-deg drugs diminish rapidly following a single injection, daily intravenous administration is required. Additionally, these drugs provide only minor improvements in motor disorders and are unable to significantly ameliorate motor and non-motor dysfunctions in CNS diseases over an extended period.

This experimental example explores the effective expression of the AAV vector constructed in example 1 in animal model from the following two aspects.

(1) An AAV control (control virus) encoding tat-polypeptide with a non-specific sequence of the same length is injected into the left substantia nigra of a 9-month-old mouse with Parkinson's disease (A53T mouse), while pAAV-CMV-tat-βSyn-deg-ef1a-egfp-3×flag is injected into the right substantia nigra. Pharmacologically active polypeptides are continuously produced by the in vivo cell pharmaceutical factory.

As shown in FIG. 4, the AAV vector of the present invention significantly reduced α-syn protein levels in the right substantia nigra and striatum, whereas the control virus showed no effect in the left side of the brain in the A53T mouse. GAPDH was used as internal reference. This result confirms that the drug encoded by the AAV vector and produced by brain cells is therapeutically effective.

(2) Control virus (ctl) was injected into the left striatum while pAAV-CMV-tat-βSyn-deg-ef1a-egfp-3×flag virus (deg) was injected into the right striatum of 3-month-old (3M), 6-month-old (6M), and 9-month-old (9M) A53T mice. At 3 months post-injection, the 6M, 9M, and 12M mice had significantly improved motor performance on the rotarod (FIG. 5, 5 times) and reduced anxiety in an open field (FIG. 6, 10 min) compared to age-matched control A53T mice that received ctl injection in the right striatum. It is specifically as follows:

In FIG. 5, A53T mice that received ctl injection in the left striatum and pAAV-CMV-tat-βSyn-deg-ef1a-egfp-3×flag injection in the right striatum at 3M and 6M of age demonstrated significantly increased latency to fall and distance travelled on the rotarod compared to control mice at 3 months post-injection. However, tat-βSyn-deg had no impact on motor dysfunction in 9M A53T mice due to extensive death of dopaminergic neurons in the substantia nigra prior to intervention. These findings demonstrate that the tat-βSyn-deg drug mediates toxic protein degradation and is more suitable for ameliorating motor dysfunction in early- to mid-stage PD.

In FIG. 6, A53T mice that received ctl injection in the left striatum and pAAV-CMV-tat-βSyn-deg-ef1a-egfp-3×flag injection in the right striatum at 3M, 6M, and 9M of age demonstrated significantly increased time spent in the center of an open field compared to control mice at 3 months post-injection. This indicates that the tat-βSyn-deg drug can significantly improve non-motor dysfunction, which is less dependent on dopaminergic neurons in the substantia nigra, making it suitable for the treatment of late-stage PD.

The above experimental results not only prove the feasibility of the technology provided by the present invention, but also confirms that the AAV vector drug of the present invention can significantly improve motor and non-motor dysfunction in mice with Parkinson's disease. The AAV vector drug of the present invention provides long-term therapeutic effects after a single administration. It addresses the limitation of conventional AAV vector drugs, which are only generated in transfected cells, by effectively crossing the cell membrane and exerting therapeutic effects across multiple brain regions. The AAV vector drug of the present invention overcomes the challenges of traditional natural polypeptide drugs, including rapid degradation that hinders the maintenance of effective drug concentrations, resulting in poor efficacy and limited improvements in motor and non-motor dysfunctions.

1. An adeno-associated virus (AAV) vector containing a nucleic acid fragment that encodes a tat-degradation peptide.

2. The AAV vector of claim 1, wherein the degradation peptide is a peptide carrying a deg tag or a CTM tag that mediates endogenous protein degradation; preferably, the amino acid sequence of the deg tag is as shown in SEQ ID NO. 1; SEQ ID NO. 1: RRRG; preferably, the amino acid sequence of the CTM tag is as shown in SEQ ID NO. 2; SEQ ID NO. 2: KFERQ.

3. The AAV vector of claim 1, wherein the amino acid sequence of the tat is as shown in SEQ ID NO. 3; SEQ ID NO. 3: YGRKKRRQRRR; preferably, the nucleic acid sequence encoding tat is as shown in SEQ ID NO. 4; SEQ ID NO. 2: TATGGCAGGAAGAAGCGGAGACAGCGACGAAGA.

4. The AAV vector of claim 1, wherein the degradation peptide is βSyn-deg; and the amino acid sequence of βSyn-deg is as shown in SEQ ID NO. 5; SEQ ID NO. 5: YGRKKRRQRRRRTKSGVYLVGRRRG; preferably, the nucleic acid sequence encoding βSyn-deg is as shown in SEQ ID NO. 6; SEQ ID NO. 6: CGTACTAAATCTGGTGTTTATTTGGTTGGTCGACGACGAGGC.

5. The AAV vector of claim 1, wherein the degradation peptide is βsyn-CTM; and the amino acid sequence of βsyn-CTM is as shown in SEQ ID NO. 7; SEQ ID NO. 7: YGRKKRRQRRRRTKSGVYLVGKFERQ.

6. The AAV vector of claim 1, wherein the AAV vector further comprises a promoter sequence; preferably, the promoter is a CMV promoter or a cell-specific promoter; preferably, the cell-specific promoter is EF1a or hSyn.

7. The AAV vector of claim 1, wherein the serotype of the AAV vector is AAV9, AAV2, AAV5 or AAV8.

8. Use of the AAV vector of claim 1 in preparing a drug for widespread and long-term intervention in CNS diseases.

9. A drug comprising the AAV vector of claim 1 and pharmaceutically acceptable excipients.

10. The drug of claim 9, wherein the drug is an injection; preferably, the drug is an intracerebral injection.
